(19) United States
(12) Patent Application Publication
(10) Pub. No.: US 2011/0004070 A1
(43) Pub. Date: Jan. 6, 2011

(54) DEVICE FOR COORDINATING PATIENT CARE IN HOSPITALS, NURSING HOMES, DOCTORS OFFICES OR THE LIKE AND METHOD FOR OPERATING SUCH A DEVICE

(75) Inventors: Max Rist, Pfaffenhofen (DE); Annett Wendorf, Dachau (DE); Hansjörg Waibel, Daueclin (NZ)

Correspondence Address: William D Lee Jr;McNair Law Firm, P O Box 447, Greenville, SC, 29302, US

(73) Assignees: ANNETT WENDORF; HANSJORG WAIBEL

(21) Appl. No.: 12/734,857

(22) PCT Filed: Dec. 2, 2008
(86) PCT No.: PCT/EP2008/010198

§ 371 Date: Aug. 30, 2010

(30) Foreign Application Priority Data
Dec. 4, 2007 (DE) 10 2007 058 640.1

(51) Int. Cl. A61B 5/00

(52) U.S. Cl. 600/300

(57) ABSTRACT

The invention relates to a device for coordinating patient care in hospitals, nursing homes, doctors offices or the like, comprising a plurality of cross-departmental medical care stations (8, 9) that are combined into at least one care group (12, 13, 16, 17), each being stationary in different care regions (1 to 6), wherein at least one mobile care station support unit (10, 11, 14, 15) that is independent from the care groups is associated with each care group (12, 13, 16, 17). Furthermore, each care group (12, 13, 16, 17) is provided with a separate, particularly wireless communication device, with which communication exclusively within each care group (12, 13, 16, 17) and thus independent from communication within a possibly existing other care group (12, 13, 16, 17) can be carried out. A radio device is preferably provided as the communication device. By means of the particular wireless communication with the communication device associated with each care group (12, 13, 16, 17), the mobile care station support units (10, 11, 14, 15), the number thereof being smaller than the number of the stationary medical care stations (8, 9) of said care group (12, 13, 16, 17), can be directed as a function of predetermined care parameters to the stationary medical care stations (8, 9) that require support via the communication device.

### FIELD OF THE INVENTION

The invention relates to a device for coordinating patient care in hospitals, nursing homes, doctors offices or the like including a wireless communication means for such a device and a method for operating such a device.

### BACKGROUND

It is generally common particularly in hospitals with multiple and separate operating theaters that, as shown schematically, for example, in FIG. 1, a stationary operating table 101 is arranged around two functionally different, stationary medical care stations 102 and 103, for example, for the duration of patient care, whereby the stationary care station 102 represents a surgical care station, for example, while the stationary care station 102 represents an anesthesia care station. These care stations are operated and led by an anesthesiologist or nurse anesthetist or the like or a surgeon or surgical instrument nurse or the like. The anesthesia care station 103 as well as the surgery care station 102 are associated in the operating theater 100 with mobile support units 104 and 105 in each case, which make available, for example, instruments, prostheses and/or any other required operating materials as well as any necessary manpower within the scope of patient care. These support units 104 and 105 are each associated with a nurse who operates and leads the support units. As schematic FIG. 1 clearly indicates, all operating theaters 100 through 500 are identically constructed so that a support unit is provided for each stationary care station in every operating theater.

These care station support devices must be available during patient care; however, they are not needed for the entire duration of patient care and are thus extremely costly. Particularly in hospitals, these support devices are not in use 50% of the time with regard to patient care. The result is high supply costs and thus overall high maintenance costs.

Conversely, it is the task of the present invention to provide a device for coordinating patient care in hospitals, nursing homes, doctors offices or the like, a wireless communication device for such a device as well as a method for operating such a device, whereby operating costs in care areas are reduced and patient care can therefore be optimized particularly with regard to technical costs.

### SUMMARY OF THE INVENTION

This task is achieved with regard to a device having wireless communication means by the method for operating the device. Beneficial embodiments of the invention are each an object of the attached claims and sub claims.

According to the present invention a plurality of cross-departmental medical care stations are combined into at least one care group, each being stationary in different care regions, wherein at least one mobile, cross-departmental care station support unit is associated with each care group. Furthermore, each care group is provided with a separate, particularly wireless communication device with which communication exclusively within each care group can be carried out. As a result the stationary medical care stations may be either functionally different or functionally similar stationary medical care stations, i.e. they may be optionally combined in principle.

With such a device according to the present invention for coordinating patient care in hospitals, nursing homes, doctor offices or the like, the number of care station support units can be beneficially reduced so that the number of care station support units of each care group may be smaller than the number of stationary care stations within said care group. Therefore, by means particularly of the wireless communication with the communication device associated with each care group, the mobile care station support units, the number thereof being smaller than the number of the stationary medical care stations of said care group, can be directed via the communication device to the stationary medical care stations that require support in order to provide the requested support, for example, materials, instruments or the like.

This means, therefore, that multiple cross-departmental, particularly functionally similar stationary medical care stations share one or more support units so that the number of care station support units can be beneficially reduced using the present invention concept. Operating costs for patient care are reduced as a result in the care regions, particularly operating theaters, which leads to considerable cost-savings and cost optimization in patient care without comprising quality. Such cost-saving results in considerable savings potential, particularly in a multitude of hospitals with operating theaters and makes a considerable contribution to the economically effective operation of a hospital in the sense of modern hospital management requirements without comprising quality in patient care.

According to a preferred concrete embodiment of the invention concept, multiple care regions are each associated with at least two different stationary medical care stations, whereby with regard to different care regions multiple functionally similar stationary medical care stations are provided, each forming a cross-departmental care group. Each care group is in turn associated with at least one mobile care station support unit that is independent from the care groups. Furthermore, each care group is provided with a separate, particularly wireless communication device with which communication exclusively within each care group and thus independent from communication within another care group is possible and can be carried out. The communication devices are designed in such a way that in life threatening emergency situations, for example, communication between the individual care groups is possible in that, for example, in connection with radio devices as the communication devices a radio frequency is predefined, which enables corresponding cross-care group communication as an emergency frequency.

In principal, any communication device that enables high-quality, understandable and essentially interference-free communication within the respective care group may be used as a technical communication device for communication within a care group. This includes particularly communication equipment or devices also designed, independent of the high communication quality, in such a way that they do not impair the use or activity of or in connection with the support unit as is beneficial, for example, with wireless communication devices. A mobile radio telephone is therefore particularly recommended according to an embodiment of the invention with regard to the use of a radio device as the said wireless communication device. This also expressly includes the use of a wireless local area network (WLAN). These radio devices are developed and laid out beneficially so that the different care groups may send and receive on different frequencies or in different radio frequency areas or bands. These radio devices may also be operated, for example, in intercommunication, in so-called half-duplex operation, or also preferably with two-way communication in so-called full-duplex operation. While in intercommunication and half-duplex operations all participants within a care group send and receive on the same frequency so that only one participant may send a signal while others listen, two-way communication allows sending and receiving on different frequencies so that communication similar to telephone communication is possible.

According to an additional concrete design of the radio device, it is provisioned to include in each care station or support unit a securable headset with earphones and microphone. This allows the operator of the individual care stations or support units to establish a communication link with high speech quality without hindering the field of vision or mobility of the arms.

The radio devices of the individual cross-departmental medical care units or groups can thereby either be adjusted to different frequency ranges from the start or alternatively be designed as adjustable to different frequency ranges.

According to an additional preferred design of the device according to the invention, it is provided that the number of mobile care station support units within a care group are predefined in such a way that a single mobile care station support unit is associated with two or three or four functionally similar stationary medical care stations within the respective care group. With such a numerical restriction, the aforementioned care benefits and cost reductions can be achieved according to a concrete design.

Furthermore, it can be beneficially provided that each cross-departmental care group has a predefined number, preferably between two and five, of stationary, functionally similar medical care stations so that if the number of total available stationary functionally similar medical care stations per care group exceeds this predefined number, the remaining stationary, functionally similar medical care stations of one or more additional care groups are associated as functionally similar care groups with regard to the included care regions. Beneficially, a plurality of functionally similar care groups may be formed independent of each other as a result, which in each case are equipped with different, particularly wireless communication devices that enable communication exclusively within each of the respective care groups and are therefore isolated from a communication within all other functionally dissimilar and functionally similar care groups. Such a numerical restriction of the care station participants within a care group guarantees greater communication clarity within the scope of care operations while maintaining technical operability in terms of care and with regard to support units as well as technical ease of use without compromising quality.

It is provided according to an additional preferred embodiment of the invention that all isolated communicating care groups, which are each associated with a group communication device, can be overlaid with at least one central coordinating communication device that preferably establishes a communication connection with each of the individual care groups. Preferably, such a central coordinating communication device can connect with the individual care groups and vice versa and the individual care groups can be connected with the central coordinating device, which is associated with the coordinating communication device. On the part of the coordinating device this may be the preferable case for individual, grouped or all care groups. In the preferred case that the central coordinating communication device is also the radio device, it can be provided that it sends and receives, particularly selectively sends and receives, over all radio frequencies or frequency ranges.

The invention concept can be applied in different areas, particularly in hospitals, nursing homes and doctors offices. Especially preferred, however, is use of the concept according to the invention in hospitals that manage operating rooms and operating theaters as care regions. These care regions may thereby be different and separate spaces accessible by corridors for example. Alternatively, the individual care regions may also represent separated areas in large spaces.

Particularly in connection with hospital operating theaters, it is provided according to a preferred concrete embodiment of the invention that the stationary medical care stations are associated in each care area with a stationary operating area, particularly an operating table.

The term “stationary”, as used with regard to the present invention concept in connection with the stationary medical care stations as well as the stationary operating area, should in the sense of the present concept idea essentially be understood as care stations or operating areas arranged in a stationary fashion, for example, for the duration of patient care, particularly an operation. This does not mean, however, that the care stations or operating area must be arranged as stationary in a care area, for example an operating theater, at times when patient care is not being performed; in fact, the stationary care stations and the stationary operating area in the sense of the present invention concept may be mobile devices or units that can be moved to or secured at other locations at any time, particularly when patient care is not being performed.

Furthermore, the term “functionally similar” in the sense of the present invention concept should be understood as
a) medically, the same specialist area, for example anesthesia or surgery to name only a couple examples, associated with the care stations and care station support units that with regard to different care regions, for example (e.g. operation theaters), execute different activities or services,
or
b) care stations and care station support units that are medically supplementary or cooperative for a certain type of care or partial care and which, if applicable, may also be associated with medically different specialist areas.

According to an additional preferred embodiment of the present invention, it is provided that the at least one mobile care station support unit that is independent from the care groups is associated with a monitoring and/or control device, which can be called up and/or displayed and/or monitored using the data from care regions associated with the at least one care station support unit, particularly from the at least one stationary care station that is associated with the care groups in the associated care regions. Transferred data particularly concerns data that is at least specific to each care group. It can also be provided as an alternative or in addition to this data transfer that data, particularly data specific to care groups and/or particularly data for the at least one stationary care station, which is particularly associated with a care group in the associated care areas, be entered via the monitoring and/or control device. With such a preferred version of the invention, a plurality of different care regions can be monitored and supervised by a doctor, for example, outside of the respective care regions.

A concrete embodiment of the invention is the monitoring and/or control device, which displays care data images using at least one piece of image display equipment and/or audibly plays back care data generated by at least one piece of sound reproducing equipment depending on the care group. As a result, monitor displays from equipment that is arranged in the care area for example, and/or images from patient care, particularly an operation in an operating theater as well as patient sounds outside of the respective care regions can be easily played back and monitored. Preferably a monitoring and/or control device with at least a camera and/or at least a microphone is coupled in the individual care regions. The coupling with this equipment is wireless preferably. A line connection is also possible in principle in this case.

It is preferred thereby that the monitoring and/or control device is formed in the area outside of the respective associated care regions by at least one installed stationary or alternatively one mobile device. For example, such a monitoring and/or control device can be arranged in the corridor area of an operating theater, particular in a sterile corridor.

The monitoring and control devices of different care groups are preferably coupled for technical data in order to enable data exchange across care groups. Alternatively or additionally, the monitoring and/or control device can also be coupled with a patient care and/or hospital management program, whereby data specific to patient care can be made accessible in those areas, for example, that are removed from the respective care regions yet require data specific to patient care, just to name one example.

### DESCRIPTION OF THE DRAWINGS

The invention will be subsequently clarified by referring to the drawings:

Shown are:

FIG. 1 a schematic example of a device for patient care in hospitals according to the current state of technology, and

FIG. 2 a schematic example of a device according to the invention for coordinating patient care in hospitals.

### DETAILED DESCRIPTION

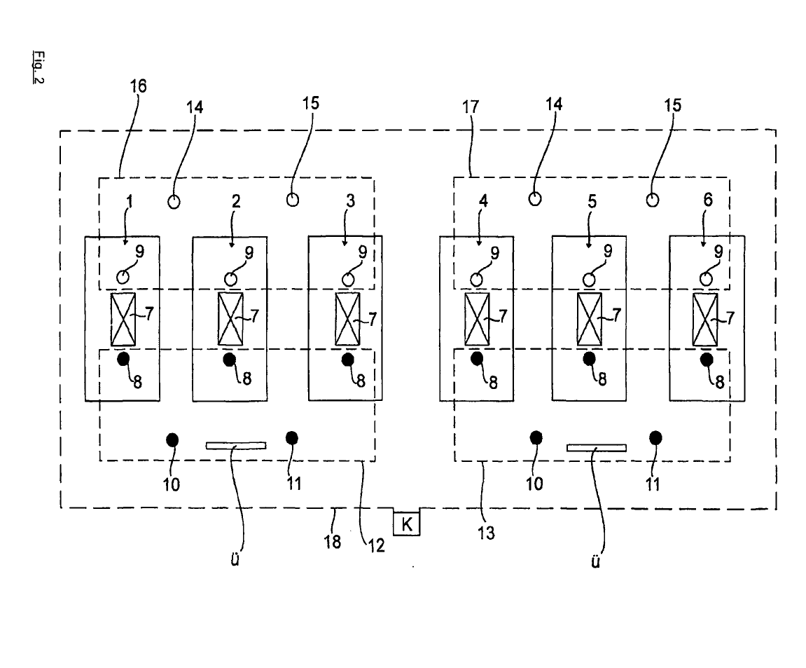

FIG. 2 shows a schematic example of a configuration of six operating theaters 1 to 6 in a hospital, whereby each operating theater, as explained using the example of operating theater 1, is associated with a stationary operating table 7, which in turn is associated with a stationary anesthesia station 8 as well as a stationary surgery care station 9, which is shown here as an example as forming two functionally different (anesthesia, surgery) stationary medical care stations.

As derived from the schematic representation in FIG. 2, the three functionally similar anesthesia care stations 8 in operating theaters 1 to 3 form, together with the two associated anesthesia support units 10, 11 associated with operating theaters 1 to 3 and shown here as examples, a so-called anesthesia care group 12, which is expressed with the dotted enclosure of the individual care stations and support units. Analogously, the anesthesia care stations 8 of operating rooms 4 through 6, together with these associated anesthesia support units 10, 11, form a second anesthesia care group 13 for operating theaters 4 to 6.

Likewise, the surgery care stations 9 of operating theaters 1 to 3 and operating theaters 4 to 6 analogously each form, together with the two surgery support units 14, 15 associated with operating theaters 1 to 3 and operating theaters 4 to 6 and also shown here as examples, separate surgery care groups 16, 17, which are also identified by the dotted enclosure.

Each of the two anesthesia care groups 12, 13 and both surgery care groups 16, 17 are associated with radio equipment not shown here, for example WLAN based radio equipment, which includes a radio device for each care station and each support unit, particularly a headset for the operator of the care stations or support units, who are doctors or nurses. Each radio device sends and receives on a different radio frequency so that with regard to the individual care group communication can be conducted exclusively within said care group and is therefore isolated from communication within a different care group.

If the surgery care station 9 in operating theater 1 requires the support of a support unit it sends notification of the corresponding requirement via the radio equipment associated with this surgery care group 16, for example, a requirement for instruments, prostheses or specialist help or the like so that as a result, for example, the surgery support unit 14 of this surgery care group 16 can support or supply the surgery care station 9 in operating theater 1 correspondingly. In the case that the surgery care station 9 in the operating theater 2, for example, simultaneously notifies of a requirement, support is then guaranteed by way of the second surgery support unit 15. Once the surgery support unit 14, for example, has ended its support of the surgery care station 9 in the first operating theater 1, it is once again available, for example, for support of the surgery care station 9 in the operating theater 2.

Analogously, this is the same for other care groups 12, 13, 17, but is not portrayed here in detail, whereby the radio frequencies of the care groups differ.

The dotted enclosures of the individual care groups of FIG. 2, which are mere examples, simultaneously represent here in each case symbolic radio clouds, which means here that notifications can be sent and received via a certain predefined radio frequency.

As is additionally derived from the schematic representation in FIG. 2, a central coordinating device K can be additionally provided that is also equipped with a radio device that sends and receives signals via all frequency bands of all care groups 12, 13, 16, 17 corresponding to the dotted enclosure for the purpose of contacting or being contacted by certain individual, multiple or all individual care groups.

In connection with both anesthesia groups 12, 13, only a schematic example of a monitoring and/or control device M associated with the anesthesia units 10, 11 outside of the operating theaters 1 to 6 is shown here. This monitoring and/or control device M of the anesthesia care group 12 is, for example, coupled with at least one camera in the operating theaters 1 to 3 or with equipment in the anesthesia care stations 8 for the purpose of signal transfer in such a way that the desired data, or in the aforementioned example the camera image data, can be called up, displayed and monitored outside of the operating theaters 1 to 3 using the monitoring and/or control device M, namely by the anesthesia support units 10, 11. The same applies analogously for the monitoring and/or control device M of the anesthesia care group 13 without going into greater detail here.

With such a monitoring and/or control device, the anesthesia support units 10, 11 can comfortably follow the operation in the operating theaters 1 to 3 outside of the respective operating theater 1 to 3 and do so for a specific group or for all groups, whereby information from the operation is received as to whether an unnoticed support requirement has been entered by the anesthesia support unit 10, 11 in one of the operating theaters 1 to 3. In such a case, the anesthesia support unit 10 or 11 can then adjourn in the respective operating theater or also issue commands or instructions via the radio device to the corresponding specific care group.

Although only the anesthesia support units 10, 11 are displayed here for this purpose, this system of monitoring and control devices M easily transfers to the surgery care groups 16, 17 as well.

As previously indicated, image data of the progress of the operation in the operating theaters can be transferred using a camera for example. Likewise, patient sounds as well as dialogue, particularly dialogue from the unassociated care groups, can also be heard.

Such a monitoring and/or control device results in an increase in flexibility and effectiveness of patient care, particularly in connection with hospitals that operate multiple parallel operating theaters.

### DESCRIPTION

### Device for Coordinating Patient Care in Hospitals, Nursing Homes, Doctors Offices or the Like and Method for Operating Such a Device

The invention relates to a device for coordinating patient care in hospitals, nursing homes, doctors offices or the like according to the preamble of claim 1, a wireless communication device for such a device according to the preamble of claim 19 and a method for operating such a device according to the preamble of claim 23.

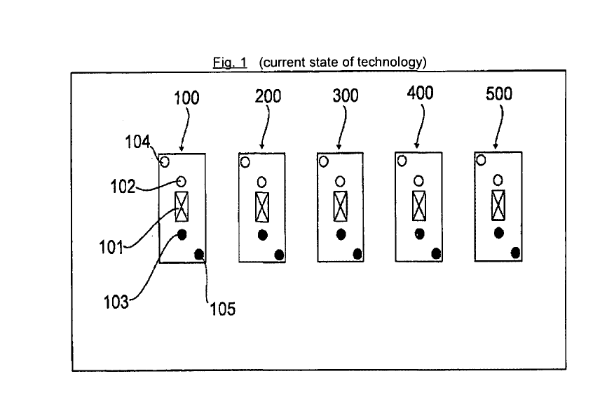

It is generally common particularly in hospitals with multiple and separate operating theaters that, as shown schematically, for example, in FIG. 1, a stationary operating table 101 is arranged around two functionally different, stationary medical care stations 102 and 103, for example, for the duration of patient care, whereby the stationary care station 102 represents a surgical care station, for example, while the stationary care station 102 represents an anesthesia care station. These care stations are operated and led by an anesthesiologist or nurse anesthetist or the like or a surgeon or surgical instrument nurse or the like. The anesthesia care station 103 as well as the surgery care station 102 are associated in the operating theater 100 with mobile support units 104 and 105 in each case, which make available, for example, instruments, prostheses and/or any other required operating materials as well as any necessary manpower within the scope of patient care. These support units 104 and 105 are each associated with a nurse who operates and leads the support units. As schematic FIG. 1 clearly indicates, all operating theaters 100 through 500 are identically constructed so that a support unit is provided for each stationary care station in every operating theater.

These care station support devices must be available during patient care; however, they are not needed for the entire duration of patient care and are thus extremely costly. Particularly in hospitals, these support devices are not in use 50% of the time with regard to patient care. The result is high supply costs and thus overall high maintenance costs.

Conversely, it is the task of the present invention to provide a device for coordinating patient care in hospitals, nursing homes, doctors offices or the like, a wireless communication device for such a device as well as a method for operating such a device, whereby operating costs in care areas are reduced and patient care can therefore be optimized particularly with regard to technical costs.

This task is achieved with regard to the device by the characteristics of claim 1, with regard to the wireless communication device by the characteristics of claim 19 and with regard to the method by the characteristics of claim 23. Beneficial embodiments of the invention are each an object of the related sub claims.

According to the present invention a plurality of cross-departmental medical care stations are combined into at least one care group, each being stationary in different care regions, wherein at least one mobile, cross-departmental care station support unit is associated with each care group. Furthermore, each care group is provided with a separate, particularly wireless communication device with which communication exclusively within each care group can be carried out. As a result the stationary medical care stations may be either functionally different or functionally similar stationary medical care stations, i.e. they may be optionally combined in principle.

With such a device according to the present invention for coordinating patient care in hospitals, nursing homes, doctor offices or the like, the number of care station support units can be beneficially reduced so that the number of care station support units of each care group may be smaller than the number of stationary care stations within said care group. Therefore, by means particularly of the wireless communication with the communication device associated with each care group, the mobile care station support units, the number thereof being smaller than the number of the stationary medical care stations of said care group, can be directed via the communication device to the stationary medical care stations that require support in order to provide the requested support, for example, materials, instruments or the like.

This means, therefore, that multiple cross-departmental, particularly functionally similar stationary medical care stations share one or more support units so that the number of care station support units can be beneficially reduced using the present invention concept. Operating costs for patient care are reduced as a result in the care regions, particularly operating theaters, which leads to considerable cost-savings and cost optimization in patient care without comprising quality. Such cost-saving results in considerable savings potential, particularly in a multitude of hospitals with operating theaters and makes a considerable contribution to the economically effective operation of a hospital in the sense of modern hospital management requirements without comprising quality in patient care.

According to a preferred concrete embodiment of the invention concept, multiple care regions are each associated with at least two different stationary medical care stations, whereby with regard to different care regions multiple functionally similar stationary medical care stations are provided, each forming a cross-departmental care group. Each care group is in turn associated with at least one mobile care station support unit that is independent from the care groups. Furthermore, each care group is provided with a separate, particularly wireless communication device with which communication exclusively within each care group and thus independent from communication within another care group is possible and can be carried out. The communication devices are designed in such a way that in life threatening emergency situations, for example, communication between the individual care groups is possible in that, for example, in connection with radio devices as the communication devices a radio frequency is predefined, which enables corresponding cross-care group communication as an emergency frequency.

In principal, any communication device that enables high-quality, understandable and essentially interference-free communication within the respective care group may be used as a technical communication device for communication within a care group. This includes particularly communication equipment or devices also designed, independent of the high communication quality, in such a way that they do not impair the use or activity of or in connection with the support unit as is beneficial, for example, with wireless communication devices. A mobile radio telephone is therefore particularly recommended according to an embodiment of the invention with regard to the use of a radio device as the said wireless communication device. This also expressly includes the use of a wireless local area network (WLAN). These radio devices are developed and laid out beneficially so that the different care groups may send and receive on different frequencies or in different radio frequency areas or bands. These radio devices may also be operated, for example, in intercommunication, in so-called half-duplex operation, or also preferably with two-way communication in so-called full-duplex operation. While in intercommunication and half-duplex operations all participants within a care group send and receive on the same frequency so that only one participant may send a signal while others listen, two-way communication allows sending and receiving on different frequencies so that communication similar to telephone communication is possible.

According to an additional concrete design of the radio device, it is provisioned to include in each care station or support unit a securable headset with earphones and microphone. This allows the operator of the individual care stations or support units to establish a communication link with high speech quality without hindering the field of vision or mobility of the arms.

The radio devices of the individual cross-departmental medical care units or groups can thereby either be adjusted to different frequency ranges from the start or alternatively be designed as adjustable to different frequency ranges.

According to an additional preferred design of the device according to the invention, it is provided that the number of mobile care station support units within a care group are predefined in such a way that a single mobile care station support unit is associated with two or three or four functionally similar stationary medical care stations within the respective care group. With such a numerical restriction, the aforementioned care benefits and cost reductions can be achieved according to a concrete design.

Furthermore, it can be beneficially provided that each cross-departmental care group has a predefined number, preferably between two and five, of stationary, functionally similar medical care stations so that if the number of total available stationary functionally similar medical care stations per care group exceeds this predefined number, the remaining stationary, functionally similar medical care stations of one or more additional care groups are associated as functionally similar care groups with regard to the included care regions. Beneficially, a plurality of functionally similar care groups may be formed independent of each other as a result, which in each case are equipped with different, particularly wireless communication devices that enable communication exclusively within each of the respective care groups and are therefore isolated from a communication within all other functionally dissimilar and functionally similar care groups. Such a numerical restriction of the care station participants within a care group guarantees greater communication clarity within the scope of care operations while maintaining technical operability in terms of care and with regard to support units as well as technical ease of use without compromising quality.

It is provided according to an additional preferred embodiment of the invention that all isolated communicating care groups, which are each associated with a group communication device, can be overlaid with at least one central coordinating communication device that preferably establishes a communication connection with each of the individual care groups. Preferably, such a central coordinating communication device can connect with the individual care groups and vice versa and the individual care groups can be connected with the central coordinating device, which is associated with the coordinating communication device. On the part of the coordinating device this may be the preferable case for individual, grouped or all care groups. In the preferred case that the central coordinating communication device is also the radio device, it can be provided that it sends and receives, particularly selectively sends and receives, over all radio frequencies or frequency ranges.

The invention concept can be applied in different areas, particularly in hospitals, nursing homes and doctors offices. Especially preferred, however, is use of the concept according to the invention in hospitals that manage operating rooms and operating theaters as care regions. These care regions may thereby be different and separate spaces accessible by corridors for example. Alternatively, the individual care regions may also represent separated areas in large spaces.

Particularly in connection with hospital operating theaters, it is provided according to a preferred concrete embodiment of the invention that the stationary medical care stations are associated in each care area with a stationary operating area, particularly an operating table.

The term “stationary”, as used with regard to the present invention concept in connection with the stationary medical care stations as well as the stationary operating area, should in the sense of the present concept idea essentially be understood as care stations or operating areas arranged in a stationary fashion, for example, for the duration of patient care, particularly an operation. This does not mean, however, that the care stations or operating area must be arranged as stationary in a care area, for example an operating theater, at times when patient care is not being performed; in fact, the stationary care stations and the stationary operating area in the sense of the present invention concept may be mobile devices or units that can be moved to or secured at other locations at any time, particularly when patient care is not being performed.

Furthermore, the term “functionally similar” in the sense of the present invention concept should be understood as

a) medically, the same specialist area, for example anesthesia or surgery to name only a couple examples, associated with the care stations and care station support units that with regard to different care regions, for example (e.g. operation theaters), execute different activities or services,
or
b) care stations and care station support units that are medically supplementary or cooperative for a certain type of care or partial care and which, if applicable, may also be associated with medically different specialist areas.

According to an additional preferred embodiment of the present invention, it is provided that the at least one mobile care station support unit that is independent from the care groups is associated with a monitoring and/or control device, which can be called up and/or displayed and/or monitored using the data from care regions associated with the at least one care station support unit, particularly from the at least one stationary care station that is associated with the care groups in the associated care regions. Transferred data particularly concerns data that is at least specific to each care group. It can also be provided as an alternative or in addition to this data transfer that data, particularly data specific to care groups and/or particularly data for the at least one stationary care station, which is particularly associated with a care group in the associated care areas, be entered via the monitoring and/or control device. With such a preferred version of the invention, a plurality of different care regions can be monitored and supervised by a doctor, for example, outside of the respective care regions.

A concrete embodiment of the invention is the monitoring and/or control device, which displays care data images using at least one piece of image display equipment and/or audibly plays back care data generated by at least one piece of sound reproducing equipment depending on the care group. As a result, monitor displays from equipment that is arranged in the care area for example, and/or images from patient care, particularly an operation in an operating theater as well as patient sounds outside of the respective care regions can be easily played back and monitored. Preferably a monitoring and/or control device with at least a camera and/or at least a microphone is coupled in the individual care regions. The coupling with this equipment is wireless preferably. A line connection is also possible in principle in this case.

It is preferred thereby that the monitoring and/or control device is formed in the area outside of the respective associated care regions by at least one installed stationary or alternatively one mobile device. For example, such a monitoring and/or control device can be arranged in the corridor area of an operating theater, particular in a sterile corridor.

The monitoring and control devices of different care groups are preferably coupled for technical data in order to enable data exchange across care groups. Alternatively or additionally, the monitoring and/or control device can also be coupled with a patient care and/or hospital management program, whereby data specific to patient care can be made accessible in those areas, for example, that are removed from the respective care regions yet require data specific to patient care, just to name one example.

The invention will be subsequently clarified using a diagram.

Shown are:

FIG. 1 a schematic example of a device for patient care in hospitals according to the current state of technology, and

FIG. 1 a schematic example of a device according to the invention for coordinating patient care in hospitals.

FIG. 2 shows a schematic example of a configuration of six operating theaters 1 to 6 in a hospital, whereby each operating theater, as explained using the example of operating theater 1, is associated with a stationary operating table 7, which in turn is associated with a stationary anesthesia station 8 as well as a stationary surgery care station 9, which is shown here as an example as forming two functionally different (anesthesia, surgery) stationary medical care stations.

As derived from the schematic representation in FIG. 2, the three functionally similar anesthesia care stations 8 in operating theaters 1 to 3 form, together with the two associated anesthesia support units 10, 11 associated with operating theaters 1 to 3 and shown here as examples, a so-called anesthesia care group 12, which is expressed with the dotted enclosure of the individual care stations and support units. Analogously, the anesthesia care stations 8 of operating rooms 4 through 6, together with these associated anesthesia support units 10, 11, form a second anesthesia care group 13 for operating theaters 4 to 6.

Likewise, the surgery care stations 9 of operating theaters 1 to 3 and operating theaters 4 to 6 analogously each form, together with the two surgery support units 14, 15 associated with operating theaters 1 to 3 and operating theaters 4 to 6 and also shown here as examples, separate surgery care groups 16, 17, which are also identified by the dotted enclosure.

Each of the two anesthesia care groups 12, 13 and both surgery care groups 16, 17 are associated with radio equipment not shown here, for example WLAN based radio equipment, which includes a radio device for each care station and each support unit, particularly a headset for the operator of the care stations or support units, who are doctors or nurses. Each radio device sends and receives on a different radio frequency so that with regard to the individual care group communication can be conducted exclusively within said care group and is therefore isolated from communication within a different care group.

If the surgery care station 9 in operating theater 1 requires the support of a support unit it sends notification of the corresponding requirement via the radio equipment associated with this surgery care group 16, for example, a requirement for instruments, prostheses or specialist help or the like so that as a result, for example, the surgery support unit 14 of this surgery care group 16 can support or supply the surgery care station 9 in operating theater 1 correspondingly. In the case that the surgery care station 9 in the operating theater 2, for example, simultaneously notifies of a requirement, support is then guaranteed by way of the second surgery support unit 15. Once the surgery support unit 14, for example, has ended its support of the surgery care station 9 in the first operating theater 1, it is once again available, for example, for support of the surgery care station 9 in the operating theater 2.

Analogously, this is the same for other care groups 12, 13, 17, but is not portrayed here in detail, whereby the radio frequencies of the care groups differ.

The dotted enclosures of the individual care groups of FIG. 2, which are mere examples, simultaneously represent here in each case symbolic radio clouds, which means here that notifications can be sent and received via a certain predefined radio frequency.

As is additionally derived from the schematic representation in FIG. 2, a central coordinating device K can be additionally provided that is also equipped with a radio device that sends and receives signals via all frequency bands of all care groups 12, 13, 16, 17 corresponding to the dotted enclosure for the purpose of contacting or being contacted by certain individual, multiple or all individual care groups.

In connection with both anesthesia groups 12, 13, only a schematic example of a monitoring and/or control device M associated with the anesthesia units 10, 11 outside of the operating theaters 1 to 6 is shown here. This monitoring and/or control device M of the anesthesia care group 12 is, for example, coupled with at least one camera in the operating theaters 1 to 3 or with equipment in the anesthesia care stations 8 for the purpose of signal transfer in such a way that the desired data, or in the aforementioned example the camera image data, can be called up, displayed and monitored outside of the operating theaters 1 to 3 using the monitoring and/or control device M, namely by the anesthesia support units 10, 11. The same applies analogously for the monitoring and/or control device M of the anesthesia care group 13 without going into greater detail here.

With such a monitoring and/or control device, the anesthesia support units 10, 11 can comfortably follow the operation in the operating theaters 1 to 3 outside of the respective operating theater 1 to 3 and do so for a specific group or for all groups, whereby information from the operation is received as to whether an unnoticed support requirement has been entered by the anesthesia support unit 10, 11 in one of the operating theaters 1 to 3. In such a case, the anesthesia support unit 10 or 11 can then adjourn in the respective operating theater or also issue commands or instructions via the radio device to the corresponding specific care group.

Although only the anesthesia support units 10, 11 are displayed here for this purpose, this system of monitoring and control devices M easily transfers to the surgery care groups 16, 17 as well.

As previously indicated, image data of the progress of the operation in the operating theaters can be transferred using a camera for example. Likewise, patient sounds as well as dialogue, particularly dialogue from the unassociated care groups, can also be heard.

Such a monitoring and/or control device results in an increase in flexibility and effectiveness of patient care, particularly in connection with hospitals that operate multiple parallel operating theaters.

1. Device for coordinating patient care in hospitals, nursing homes, doctors offices or the like:
a) comprising a plurality of cross-departmental medical care stations (8, 9) that are combined into at least one care group (12, 13, 16, 17), each being stationary in different care regions (1 to 6);
b) wherein at least one mobile, care station support unit (10, 11, 14, 15) that is independent from the care groups is associated with each care group (12, 13, 16, 17); and
c) whereby each care group (12, 13, 16, 17) is provided with a particularly wireless communication device, with which communication can be carried out within each care group (12, 13, 16, 17).

2. Device according to claim 1, characterized by the provision of a plurality of care regions (1 to 6) each associated with at least two functionally different stationary medical care stations (8, 9):
a) whereby with regard to different care regions (1 to 6) multiple functionally similar stationary medical care stations (8, 9) are provided, each forming a cross-departmental care group (12, 13, 16, 17):
b) whereby at least one mobile care station support unit (10, 11, 14, 15) that is independent from the care groups is associated with each care group (12, 13, 16, 17), and
c) whereby the care groups (12, 13, 16, 17) are equipped with separate, particularly wireless communication devices that enable communication exclusively within each of the respective care groups (12, 13, 16, 17) and are therefore isolated from a communication within another care group (12, 13, 16, 17).

3. Device according to claim 2, characterized by the number of care station support units (10, 11, 14, 15) of each care group beings smaller than the number of stationary care stations (8, 9) within each care group (12, 13, 16, 17).

4. Device according to claim 3, characterized by the number of mobile care station support units (10, 11, 14, 15) within a care group (12, 13, 16, 17) being predefined in such a way that a single mobile care station support unit (10, 11, 14, 15) is associated with two or three or four functionally similar stationary medical care stations (8, 9) within the respective care group (12, 13, 16, 17).

5. Device according to claim 4, characterized by each cross-departmental care group (12, 13, 16, 17) having a predefined number, preferably between two and five, of stationary, functionally similar medical care stations (8, 9) so that if the number of total available stationary functionally similar medical care stations per care group (12, 13, 16, 17) exceeds this predefined number, the remaining stationary, functionally similar medical care stations (8, 9) of one or more additional care groups (12, 13, 16, 17) are associated as functionally similar care groups for the formation of multiple functionally similar care groups (12, 13, 16, 17) that are independent of each other, which in each case are equipped with different, particularly wireless communication devices so that communication exclusively within each of the respective care groups (12, 13, 16, 17) and therefore isolated from a communication within all other functionally dissimilar and functionally similar care groups (12, 13, 16, 17) can be carried out.

6. Device according to claim 1, characterized by each communication device of a care group (12, 13, 16, 17) forming a group communication device, whereby all group communication devices can be connected or can remain connected to a communication link with at least one central coordinating communication device, which can either contact a coordinating device (K) from one of the care groups (12, 13, 16, 17) or contact using the care groups (12, 13, 16, 17), individual, grouped or all care groups (12, 13, 16, 17).

7. Device according to claim 1 characterized by the technical wireless communication devices in each case being formed of radio devices, particularly mobile radio telephone devices and/or using a WLAN.

8. Device according to claim 7, characterized by the different care groups (12, 13, 16, 17) sending and receiving on different radio frequencies or in different radio frequency areas, whereby in the case of a coordinating radio device according to claim 6, the device can send and receive over all radio frequencies or radio bands or areas.

9. Device according to claim 7, characterized by the radio devices being operable in intercommunication (half-duplex operation) or in two-way communication (full-duplex operation).

10. Device according to claim 7 characterized by the radio device having in each station or unit a securable headset with earphones and microphone.

11. Device according to claim 1, characterized by the care regions (1 to 6) being operating rooms or theaters in hospitals, doctors offices, examination rooms or the like.

12. Device according to claim 1 characterized by the stationary medical care stations (8, 9) are associated in each care area with a stationary examination or operating area (7), particularly an operating table.

13. Device according to claim 1 characterized by a first care area (1) being associated with at least one stationary anesthesia care station (8) and at least one stationary surgery care station (9), which along with a predefined number of anesthesia care stations (8) and surgery care stations (9) from other care regions (2 to 6) form at least one anesthesia care group (12, 13) and at least a surgery care group (16, 17), each associated with a predefined number of functionally corresponding mobile anesthesia or surgery support units (10, 11, 14, 15) independent from the care groups.

14. Device according to claim 1 characterized by the at least one mobile care station support unit (10, 11, 14, 15) of a care group (12, 13, 16, 17) that is independent from the care groups being associated with at least one monitoring and/or control device (M), which can be called up and/or displayed and/or monitored using the data, particularly data from the associated care regions (1 to 6) specific to patient care from the at least one care station support unit (10, 11, 14, 15) and/or from the care station (8, 9) particularly associated with the care groups and/or using data, particularly data specific to care groups and/or data that can be entered for the at least one stationary care station (8, 9) particularly associated with the care groups in the associated care regions (1 to 6).

15. Device according to claim 14, characterized by the monitoring and/or control device (M) is formed by at least one image display device that displays care data images and/or at least one sound reproducing device that audibly plays back care data.

16. Device according to claim 15, characterized by the monitoring and/or control device (M) being coupled, particularly wireless, with at least a camera and/or at least a microphone in the individual care regions (1 to 6).

17. Device according to claim 14, characterized by the monitoring and/or control device (M) being formed in the area outside of the respective associated care regions (1 to 6) by at least one installed stationary or one mobile device.

18. Device according to claim 14, characterized by the monitoring and/or control device (M) of different care groups (12, 13, 16, 17) being coupled for technical data exchange and/or the monitoring and/or the control device (M) being coupled with a patient care and/or hospital management program.

19. Wireless communication device for a device for coordinating patient care in hospitals, nursing homes, doctors offices or the like, particularly for a device according to claim 1, characterized by the communication device having a radio device, particularly a radio telephone device, which can be assigned to shared medical care units or groups (12, 13, 16, 17) in different care regions (1 to 6), particularly operating theaters, and particularly assigned in such a way that in the case of multiple care groups (12, 13, 16, 17) the radio devices of the individual cross-departmental medical care units or groups (12, 13, 16, 17) can send and receive on different radio frequencies and/or in different frequency bands.

20. Wireless communication device according to claim 18, characterized by the radio devices being adjusted or adjustable to different frequency ranges and/or being a component of a WLAN.

21. Wireless communication device according to claim 19 characterized by the radio devices being operable in intercommunication (half-duplex operation) or in two-way communication (full-duplex communication).

22. Wireless communication device according to claim 19 characterized by the radio device having a securable headset with earphones and microphone in each care station (8, 9) or support unit (10, 11, 14, 15).

23. Method for operating a device according to claim 1 characterized thereby that within a care group (12, 13, 16, 17), by means of the particularly wireless communication with this communication device associated with each care group (12, 13, 16, 17), the mobile care station support units (10, 11, 14, 15), the number thereof being smaller than the number of the stationary medical care stations (8, 9) of said care group (12, 13, 16, 17), can be directed as a function of
